(19) United States
(12) Patent Application Publication
(10) Pub. No.: US 2008/0139922 A1
(43) Pub. Date: Jun. 12, 2008

(54) Evaluation of cartilage of the hip using MRI images

(76) Inventors: Jean-Pierre Pelletier, Saint Lambert (CA); Johanne Martel-Pelletier, Saint Lambert (CA); Francois Abram, Montreal (CA); Wei Li, Montreal (CA)

Correspondence Address: KRISTOFER E. ELBING, 187 PELHAM ISLAND ROAD, WAYLAND, MA, 01778, US

(21) Appl. No.: 11/880,437

(22) Filed: Jul. 20, 2007

Related U.S. Application Data
(60) Provisional application No. 60/832,825, filed on Jul. 21, 2006.

(51) Int. Cl. A61B 5/05

(52) U.S. Cl. 600/410

(57) ABSTRACT

In one general aspect, the invention features an MRI image processing method that includes accessing a first MRI data set including two-dimensional planar MRI images of a first hip of a first patient. Each of the images is positioned with respect to a virtual axis at a different angle so as to distribute the images axially around the virtual axis. The virtual axis is defined as an axis that runs from the fovea through the femoral neck of the femur of the first patient. The method also includes segmenting bone and cartilage surfaces in the first MRI data set.

### CROSS-REFERENCE TO RELATED APPLICATION

This application claims the benefit under 35 U.S.C. 119(e) of U.S. provisional application No. 60/832,825, filed Jul. 21, 2006, which is herein incorporated by reference.

### BACKGROUND OF THE INVENTION

There has been significant interest in studying cartilage condition and thickness using Magnetic Resonance Imaging (MRI) techniques. These techniques allow for the measurement and mapping of cartilage thickness, and allow changes in cartilage to be tracked over time. Although other joints have been referred to, the overwhelming majority of studies have focused on the knee, which is a large joint that is generally heavily loaded and is often afflicted by osteoarthritis.

### SUMMARY OF THE INVENTION

Several aspects of the invention are presented in this application. These relate generally to the evaluation of the hip using MRI techniques.

Systems according to the invention can provide for precise and accurate imaging and analysis of a patient's hip cartilage. This can enhance the study and diagnosis of diseases affecting the hip.

In one general aspect, the invention features an MRI image processing method that includes accessing a first MRI data set including two-dimensional planar MRI images of a first hip of a first patient. Each of the images is positioned with respect to a virtual axis at a different angle so as to distribute the images axially around the virtual axis. The virtual axis is defined as an axis that runs from the fovea through the femoral neck of the femur of the first patient. The method also includes segmenting bone and cartilage surfaces in the first MRI data set.

In preferred embodiments, the method can further include the step of measuring a cartilage characteristic based on results of the step of segmenting. The method can further include a step of accessing a second MRI data set acquired after the first data set and including another plurality of two-dimensional planar MRI images of the first hip of the first patient, with each of the images passing through the virtual axis at a different angle so as to distribute the images axially around the virtual axis, and segmenting bone and cartilage surfaces in the second MRI data set. The method can further include spatially registering results of the steps of segmenting for the first and second MRI data sets. The method can further include comparing results of the steps of segmenting to evaluate cartilage changes. The step of spatially registering can take place for at least part of the femur that is below the neck of the femur. The method can further include comparing results of the steps of segmenting to evaluate cartilage changes. The method can further include evaluating disease progression based on the step of comparing. The method can further include evaluating a pharmaceutical effect on disease progression based on the step of comparing. The step of accessing can access a plurality of images acquired with a water excitation sequence. The step of accessing can access a plurality of MEDIC images. The step of accessing can access a plurality of images acquired with a semi-flexible antenna. The step of accessing can access plurality of images in which there is a forced internal rotation of the femoral neck. The virtual axis can bisect the MRI images. The method can further include the steps of fitting a sphere to the femoral head of the first hip of the first patient and transforming data derived from the MRI data set into a polar coordinate system that is based on the sphere fitted in the step of fitting. The method can further include expressing positions as a distance to the center of the sphere. The method can further include selectively reorienting the images in the first plurality of two-dimensional planar MRI images and assembling them into a three-dimensional image data set.

In another general aspect, the invention features an MRI image processing apparatus that includes an MRI data access module operative to access a first MRI data set including a first plurality of two-dimensional planar MRI images of a first hip of a first patient. Each of the images is positioned with respect to a virtual axis at a different angle so as to distribute the images axially around the virtual axis. The virtual axis is defined as an axis that runs from the fovea through the femoral neck of the femur of the first patient. A segmentation module is operative to segment bone and cartilage surfaces in the first MRI data set accessed by the MRI data access module.

In preferred embodiments, the apparatus can further include a registration module responsive to the segmentation module. The apparatus can further include an image comparison module responsive to at least the segmentation module. The MRI data access module can be directly responsive to an MRI acquisition system that employs a MEDIC sequence. Interconnections between elements can include intermittent connections.

In a further general aspect, the invention features an MRI image processing apparatus that includes means for accessing a first MRI data set including a first plurality of two-dimensional planar MRI images of a first hip of a first patient. Each of the images is positioned with respect to a virtual axis at a different angle so as to distribute the images axially around the virtual axis. The virtual axis is defined as an axis that runs from the fovea through the femoral neck of the femur of the first patient. The apparatus also includes means for segmenting bone and cartilage surfaces in the first MRI data set accessed by the means for accessing.

### BRIEF DESCRIPTION OF THE DRAWINGS

FIG. 1 is a block diagram of an illustrative cartilage imaging system according to the invention;

FIG. 2 is an outline drawing of a hip joint, showing a virtual axis used for imaging with the system of FIG. 1;

FIG. 3 is a flow chart outlining the operation of the system of FIG. 1;

FIG. 4 is a diagrammatic downward-facing axial cross-section of a patient outfitted with a flexible antenna, taken at the approximate level of his or her waist line while lying on his or her back;

FIG. 5 is a perspective diagram illustrating the orientation of four illustrative imaging planes with respect to the virtual axis of FIG. 1;

FIG. 6a is a bone and cartilage contour plot of the type that can be produced by the system of FIG. 1 for the top portion of a femur of a patient;

FIG. 6b is an image showing only the bone contour for the plot of FIG. 6a;

FIG. 6c is an image showing only the cartilage contour for the plot of FIG. 6a;

FIG. 6d is a bone and cartilage contour plot of the type that can be produced by the system of FIG. 1 for an acetabulum of the patient;

FIG. 6e is an image showing only the bone contour for the plot of FIG. 6d;

FIG. 6f is an image showing only the cartilage contour for the plot of FIG. 6d;

FIG. 7a is a printout of the surface of a 3D model of the head of the femur derived from data used to prepare the plots of FIGS. 6a and 6d;

FIG. 7b is a printout of the surface of a 3D model of the acetabulum cartilage corresponding to the head of the femur model shown in FIG. 7a;

FIG. 8 is a perspective diagram illustrating the orientation of a fitted spherical primitive with respect to the bone and cartilage surfaces of FIGS. 6a and 6d;

FIG. 9a is a cartilage volume map for the femoral head of the type that can be produced by the system of FIG. 1; and

FIG. 9b is a cartilage volume map for the acetabulum of the type that can be produced by the system of FIG. 1.

### DETAILED DESCRIPTION OF AN ILLUSTRATIVE EMBODIMENT

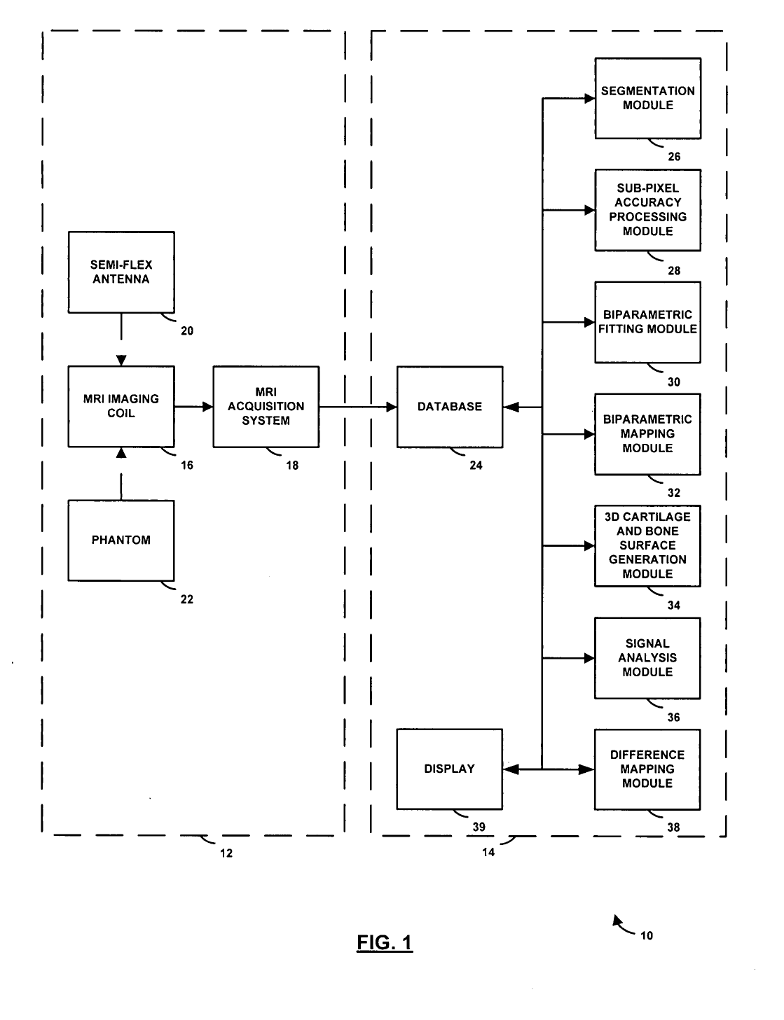

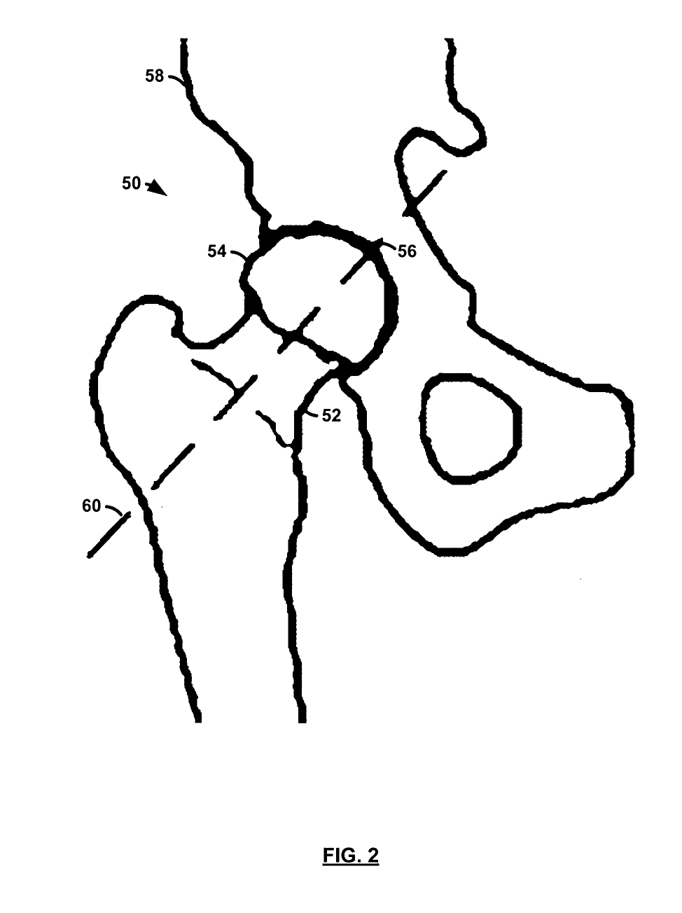

Referring to FIG. 1, an illustrative disease progression monitoring system 10 according to the invention is configured for monitoring rheumatic diseases affecting cartilage in the hip. It employs the apparatus described in U.S. Pat. No. 6,560,476, entitled “EVALUATING DISEASE PROGRESSION USING MAGNETIC RESONANCE IMAGING,” issued on May 6, 2003, and herein incorporated by reference. This apparatus has been modified to work with the hip, as described below.

The disease progression monitoring system 10 includes an acquisition subsystem 12 and a processing subsystem 14. The acquisition subsystem includes an MRI imaging coil 16 operatively connected to an MRI acquisition system 18. A semi-flexible antenna 20 that is compatible with the MRI imaging coil and a phantom 22 also form a part of the acquisition subsystem. The acquisition subsystem can include a commercially available 1.5 Tesla MRI imaging system, such as are available from Siemens AG of Munich, Germany. A suitable semi-flexible body antenna is also available from Siemens.

The processing subsystem 14 includes a database 24 that is operatively connected to the MRI acquisition system. The operative connection between the MRI acquisition system and the database can take different forms, such as a network connection or a dedicated fiber-optic link. It may also take the form of an intermittent connection, such as an e-mail link, or a physically transported high-capacity storage medium, such as an optical disk. The database can range from a collection of files for smaller research systems to more powerful and feature-rich databases for systems configured to process data for larger numbers of patients.

Also included in the processing system are a segmentation module 26, a sub-pixel processing module 28, a biparametric fitting module 30, a biparametric mapping module 32, a three-dimensional bone and cartilage surface generation module 34, a signal analysis module 36, a difference mapping module 38, and a display 39. These can all be operatively connected to the database such that they can access raw data sets received from the acquisition subsystem 12, as well as different processed versions of these data sets. Each of these modules can be implemented using special-purpose hardware, software running on a general-purpose processor, or a combination of both. In addition, while the system can be broken into the series of modules shown in FIG. 1, one of ordinary skill in the art would recognize that it is also possible to combine them and/or split them to achieve a different breakdown. In one embodiment, the modules and database are part of a larger software system that runs on one or more workstation computers outfitted with an operating system such as Microsoft's Windows® 9× or Windows NT® operating system.

In operation, referring also to FIGS. 2-5, an MRI system operator wraps the semi-flexible antenna 72 around the hip to be imaged 50, and secures the antenna in place with its strap 76 (step 100). The operator then positions the patient 70 on his or her back, with a pad 74 to support the opposite hip 51. The operator also places another pad between the patient's ankles, and positions the his or her big toes tightly together, such as by taping, to force an internal rotation of the femoral neck 52. The operator can then introduce the patient feet first into the MRI coil 16, in a direction generally parallel to a longitudinal axis of the imaging coil.

With the patient in the coil, the operator initiates a low resolution scout image to determine whether the features to be imaged are positioned at an optimal position within the coil (step 102). If not, the patient can be moved (step 104).

The operator then initiates a higher-resolution scout scan (step 106). This scan should be of a resolution that is sufficient to distinguish the features of the patient's femoral neck 52. In the present embodiment this is a 27-image scan, but the exact number of planes used is not critical.

In the second scout image, the operator determines the position of a virtual axis that runs from the fovea 56 through the femoral neck 52. The fovea is an easy-to-locate anatomic spot that is not covered with cartilage and is located where the round ligament adheres to the femoral head 54. The operator positions his or her first image in such a way that the virtual axis passes through its equator and the image is large enough to cover the femoral head, femoral neck, and part of the femur below the femoral neck (step 108).

The operator then measures the diameter of the femoral head to determine the number of images required, as discussed in more detail below. Once the operator has input this number of images into the system, he or she can initiate the acquisition (step 114).

The MRI acquisition sequence has been developed specifically for obtaining images of the hip joint 50 that include appropriate levels of information about cartilage. Unlike many sequences used for the knee, it is a two-dimensional (2D) sequence. This sequence enhances the cartilage-to-soft-tissues interface with Water Excitation, which is used to saturate the fat of the bone. The objective of this sequence is to optimally distinguish the cartilage from the bone and soft tissues.

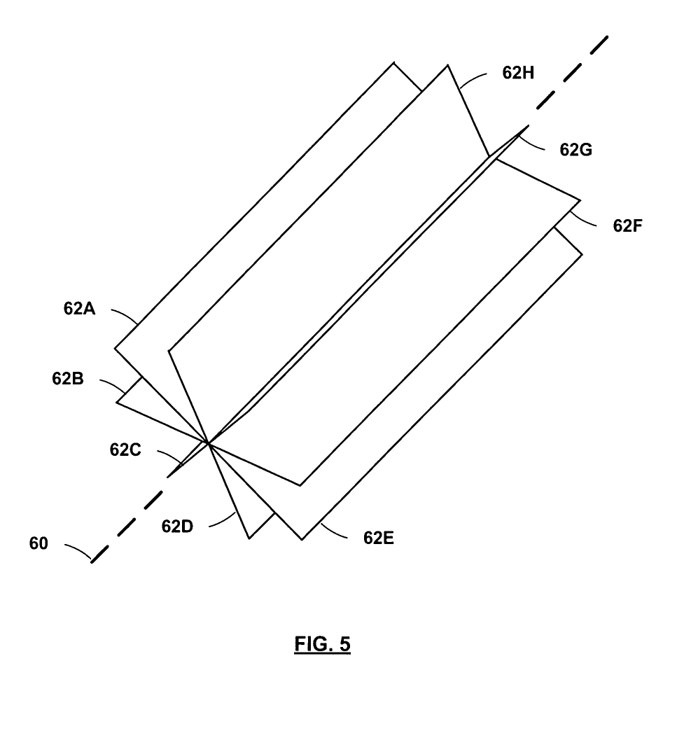

The acquisition planes 62 are not parallel to one another as has been advocated for the knee. Instead, they are disposed radially around a virtual axis 60 that runs from the fovea 56 through the femoral neck 52 (see FIG. 5). This approach obtains images of cartilage perpendicular to its surface, in order to evaluate cartilage thickness more easily, and to be able to delineate femoral cartilage from the concave acetabulum cartilage on the pelvis 58. This is accomplished in the illustrative embodiment with perpendicular images that are bisected by the virtual axis. Each image therefore covers a slice that extends from one side of the femoral head to the other, through the virtual axis. For this reason, the slices being acquired need only be rotated through 180° to acquire a full data set. Other equivalent protocols could also be developed to obtain perpendicular or at least generally perpendicular images, however, such as one in which twice as many smaller images extend radially out from the virtual axis in one direction only.

The acquisition consists of several 2D images that sample the 3D volume of the femoral head and acetabulum (the acetabulum being the articular part of the hip). Each 2D sequence can be a Multiple Echo Data Image Combination (MEDIC) sequence, which enhances the cartilage-to-soft-tissues interface with Water Excitation (WE). The objective of this sequence is to obtain the best contrast between cartilage and other tissues, including bone and soft tissues. The parameters of the MEDIC sequence are:
Water Excitation
Field of View=160 cm
Acquisition Matrix=[320,320]
TR=432 ms
TE=20 ms
Slice Thickness=3 mm
Flip Angle=59 deg.
   This sequence has been found to yield very good results. But one of ordinary skill in the art will recognize that minor changes in this sequence may still yield acceptable results, and that other types of 2D sequences may also provide adequate contrast levels in particular circumstances.

The number of acquisition planes depends on the radius of the femoral head and the spatial resolution of the MRI sequence. Too few planes will leave out information between slices. And too many planes increases the duration of the acquisition, which can tire the patient unnecessarily and increase the risk that he or she will move during the acquisition. Using too many planes can also result in oversampling artifacts. The number of planes should therefore be optimized to obtain the best distribution of voxels at the cartilage surface. This can be accomplished generally through the use of the following formula:

nₛ=πd_{f}/2tₛ

### Where:

nₛ=number of slices
π=pi
d_{f}=approximate diameter of femoral head
tₛ=slice thickness

### In trials of the technique that used a 3 mm slice thickness on a number of patients, it was found that the optimum number of planes ranged from about 28 to 42.

The star acquisition sequence used by the system acquires images that are oriented in a variety of directions with respect to the MRI coils. For this reason, it may not be possible to acquire all of the images using the same phase encoding direction. The result is that some of the images may be oriented differently with respect to each other. If this is the case, the system can selectively reorient the images (step 116) before assembling them into a three-dimensional image data set (step 118). In this embodiment, the task of reorienting the images is based on orientation information that the MRI imaging system provides in the header supplied with each image.

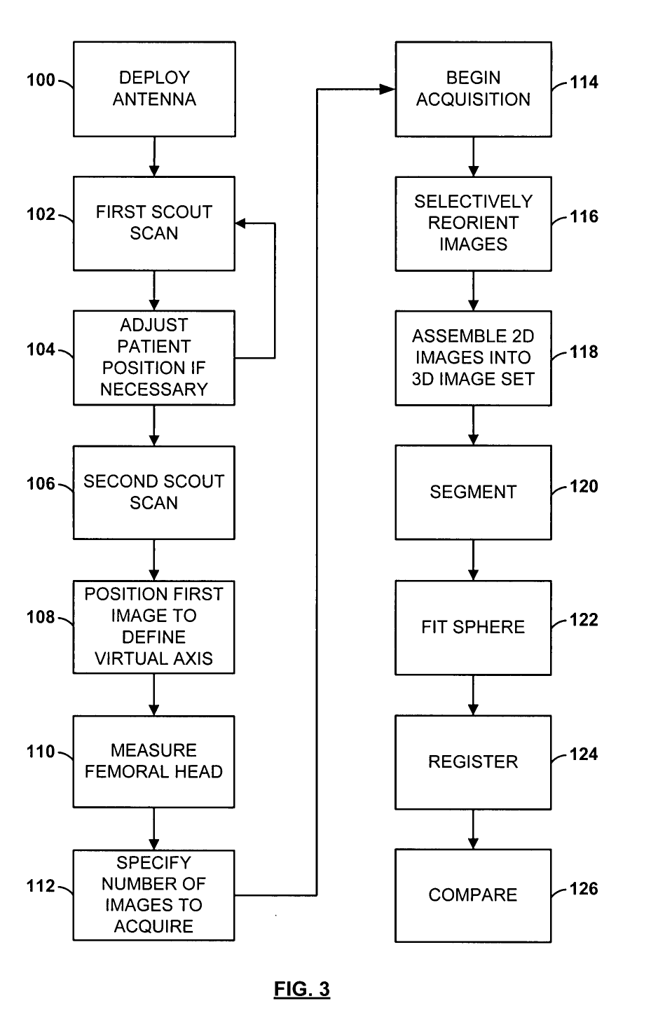

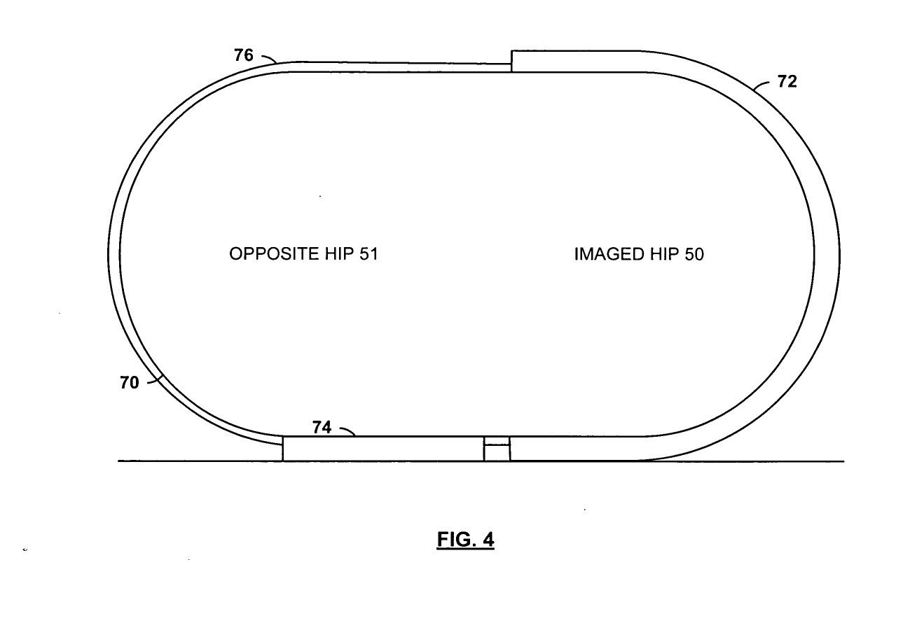

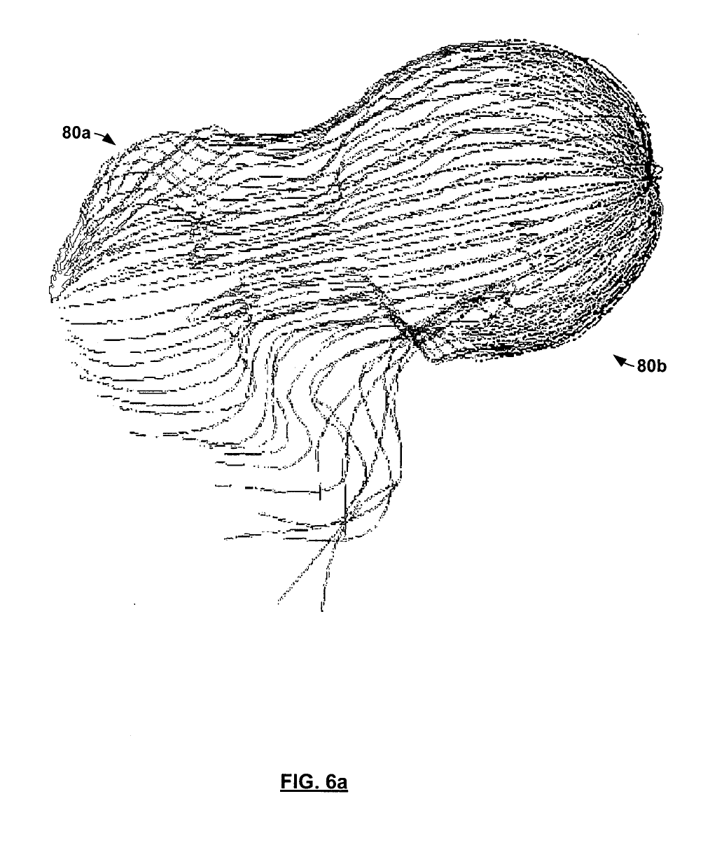

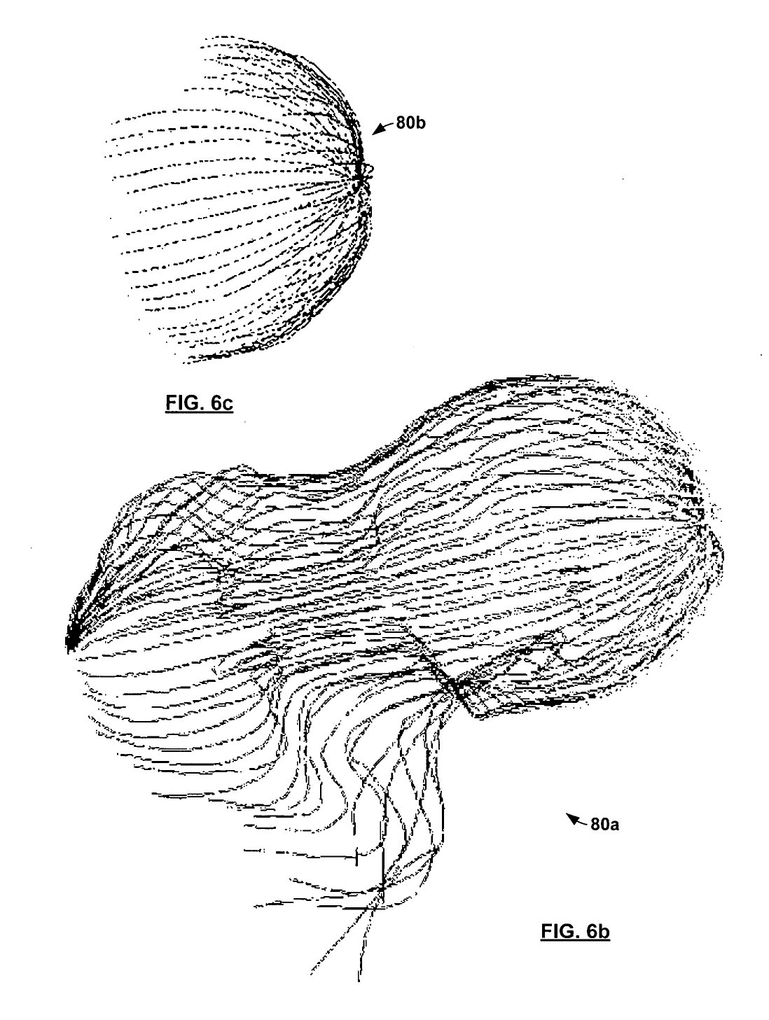

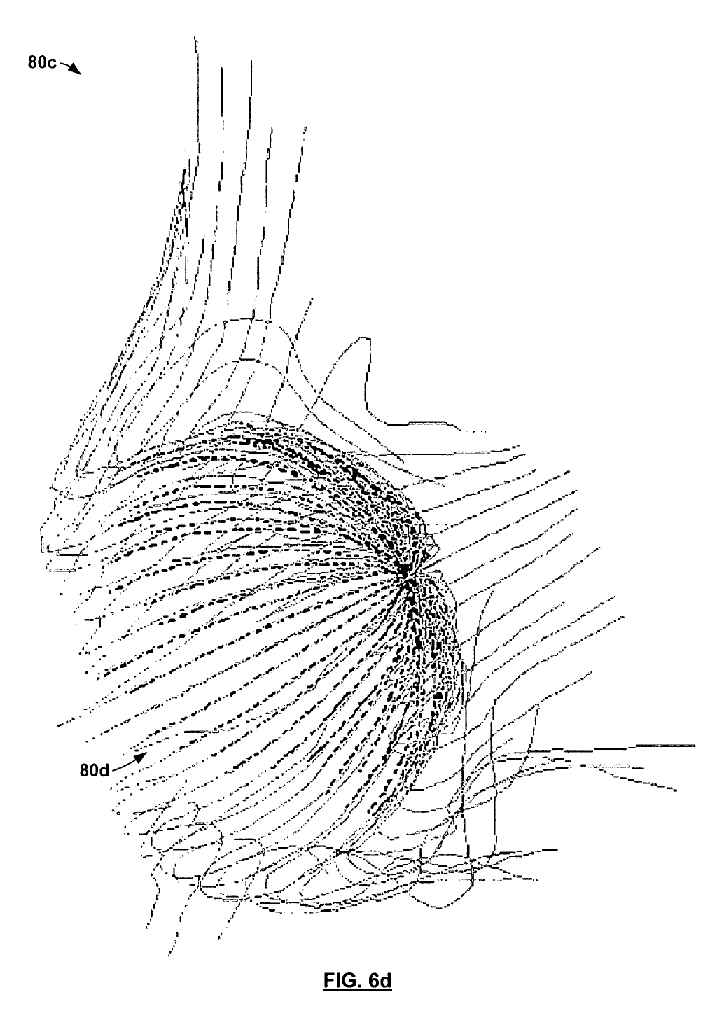

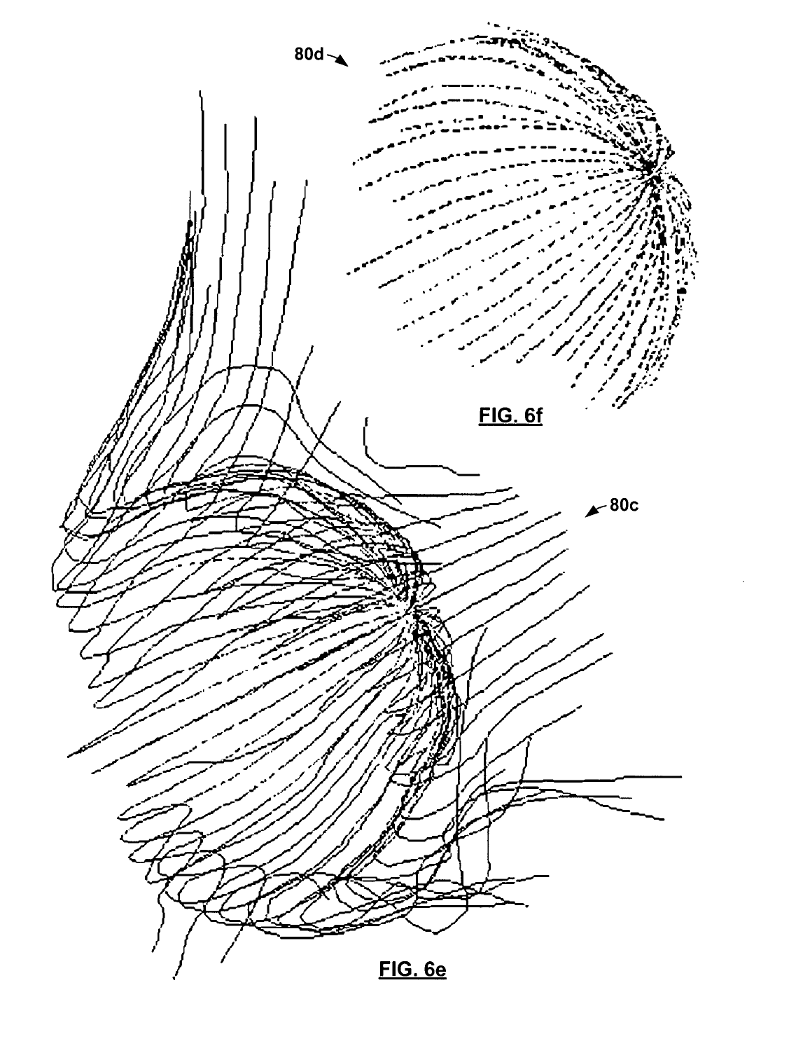

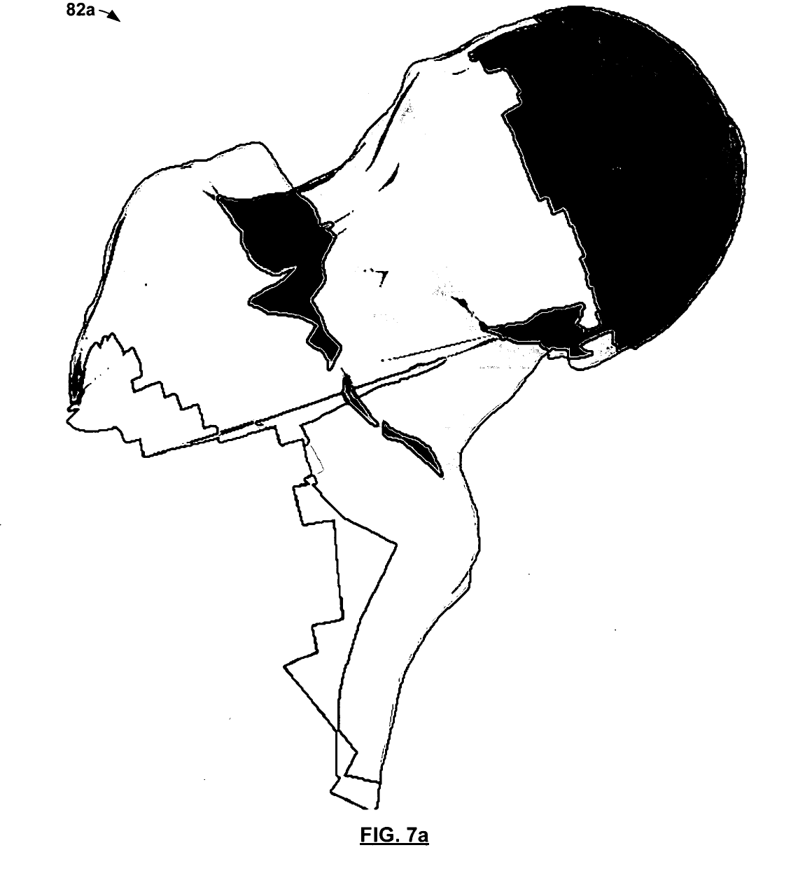

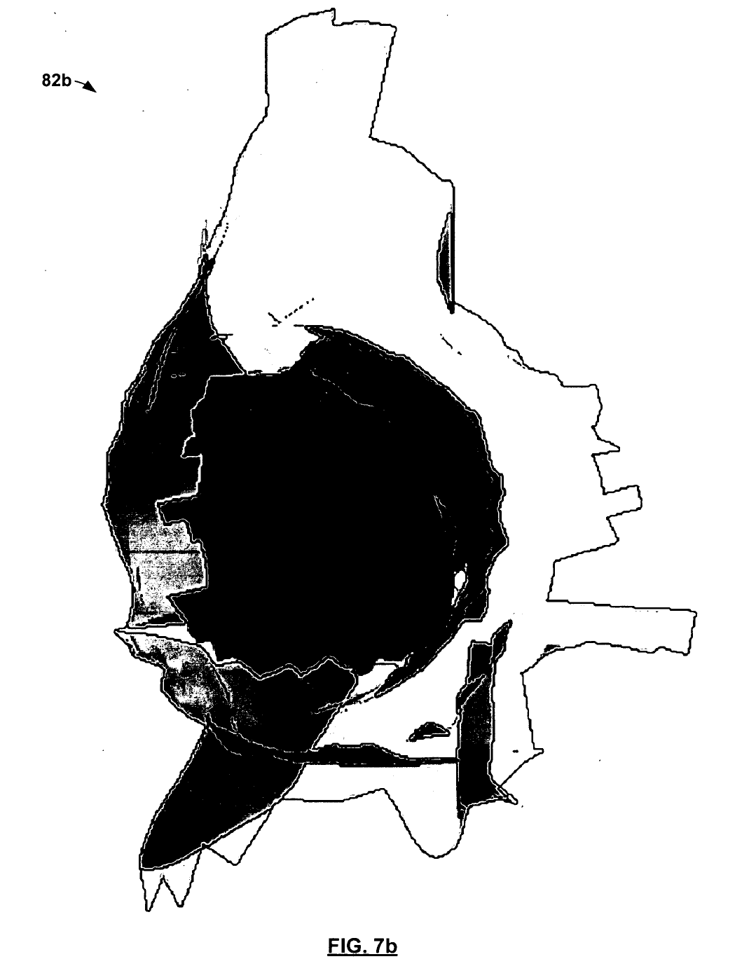

Referring to FIGS. 3-6, the bones and cartilage for the femoral head and for the acetabulum are segmented separately for each image after they are acquired, using techniques described in the above-mentioned patent (step 120). This process produces a baseline set of 3D bone contours 80a, 80c and cartilage contours 80b, 80d for the head of the femur and/or the acetabulum. A 3D model of the bone and cartilage surfaces can then be computed from these contours (see model surface printouts 82a, 82b in FIG. 7).

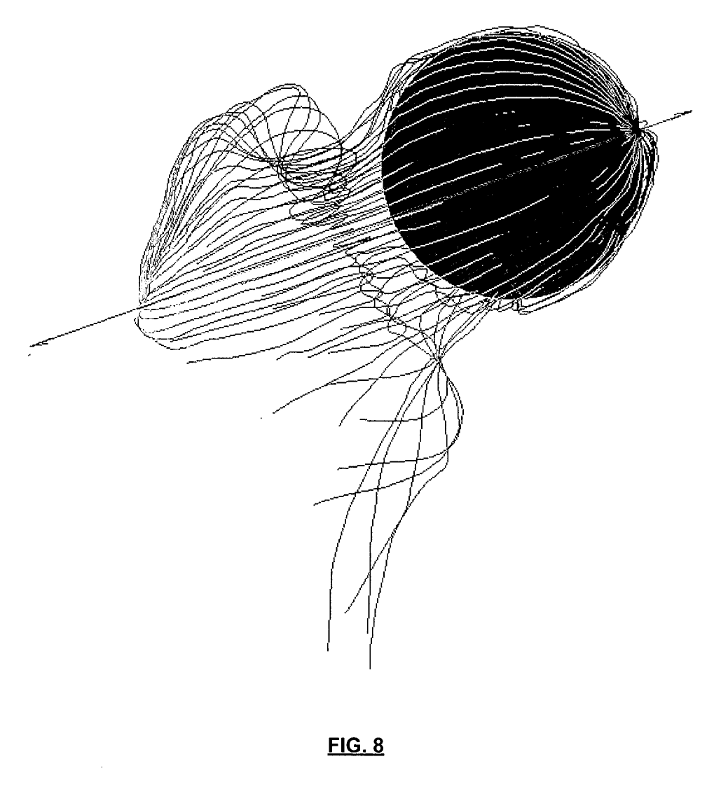

Referring also to FIG. 8, a best-fit sphere is derived for the femoral head from the segmented data (step 122). The center of this sphere represents the origin of the coordinate system that will be used on the baseline and subsequent images. This sphere is the largest sphere that will fit to the femoral head, and in this embodiment it is obtained using a robust least square optimizer. The optimizer helps to compute the best center and radius parameters with respect to selected points from the bone contours. An initialization is performed to first compute a good estimate of the position of the center and to first select a subset of all the contour points that correspond to the femoral head.

All of the surfaces in the model are then re-sampled in the polar coordinate system for use in further operations, such as measuring cartilage thickness or comparing images for different visits. With a coordinate system that is common to all visits, the computed cartilage thickness maps are comparable pixel-to-pixel, thus allowing the assessment of the progression of disease. Computations and data storage requirements can be simplified by treating the magnitude of all coordinates as a distance to the best fit sphere.

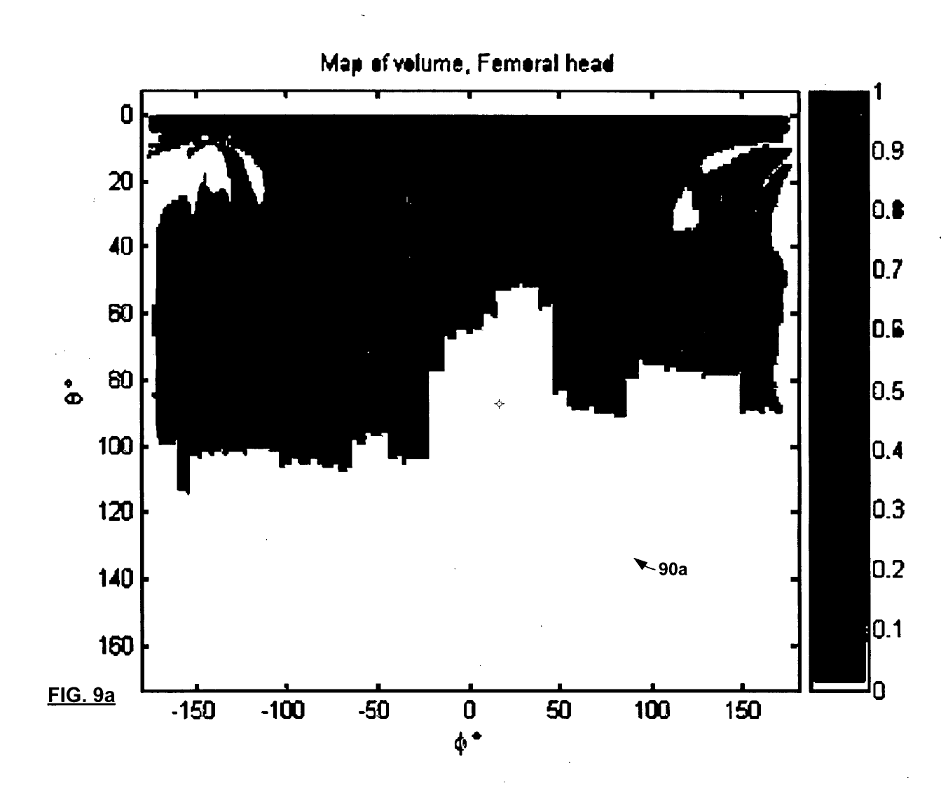

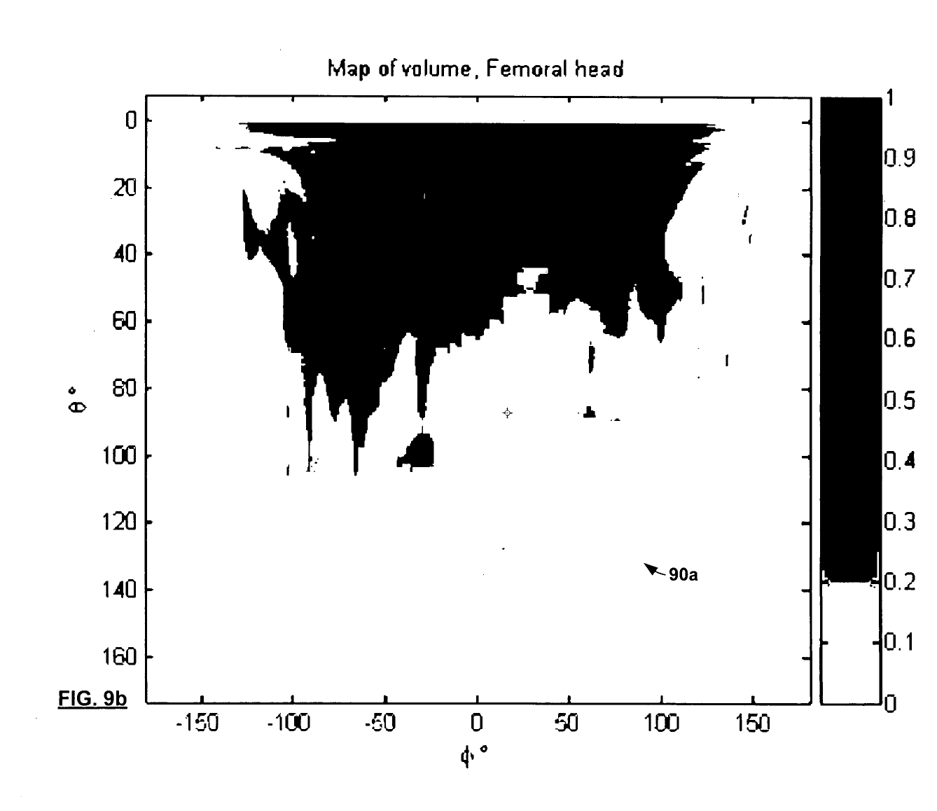

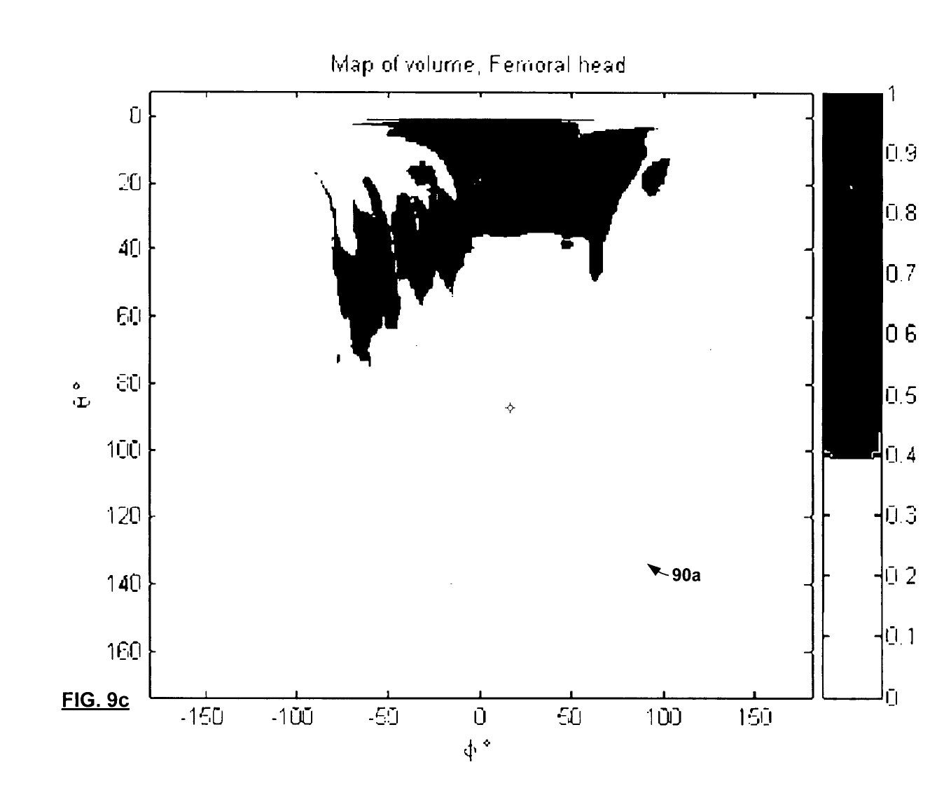

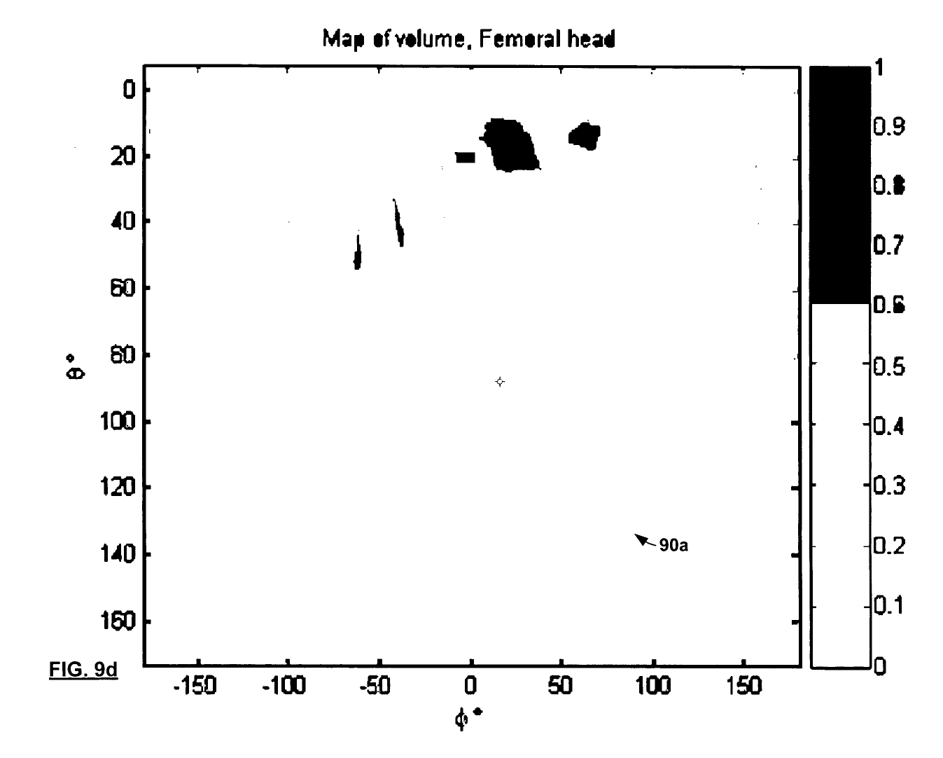

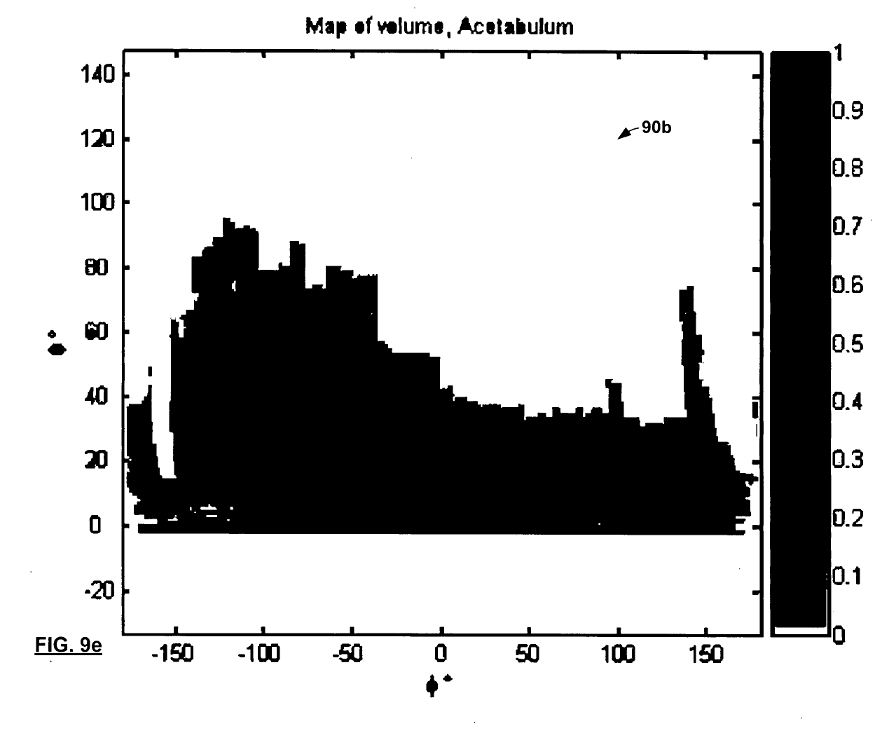

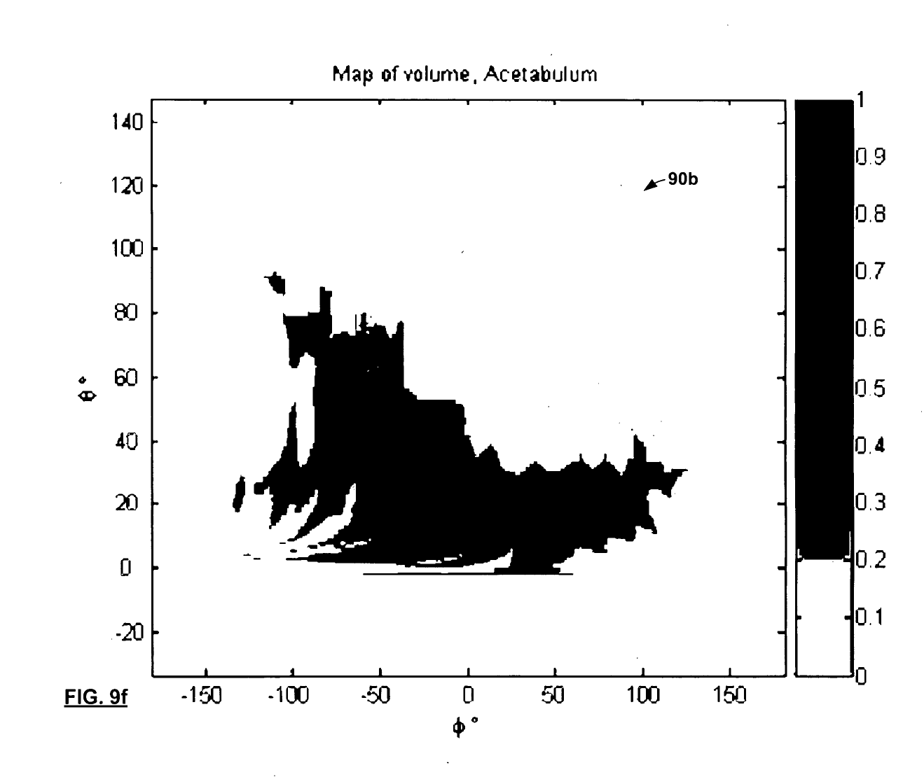

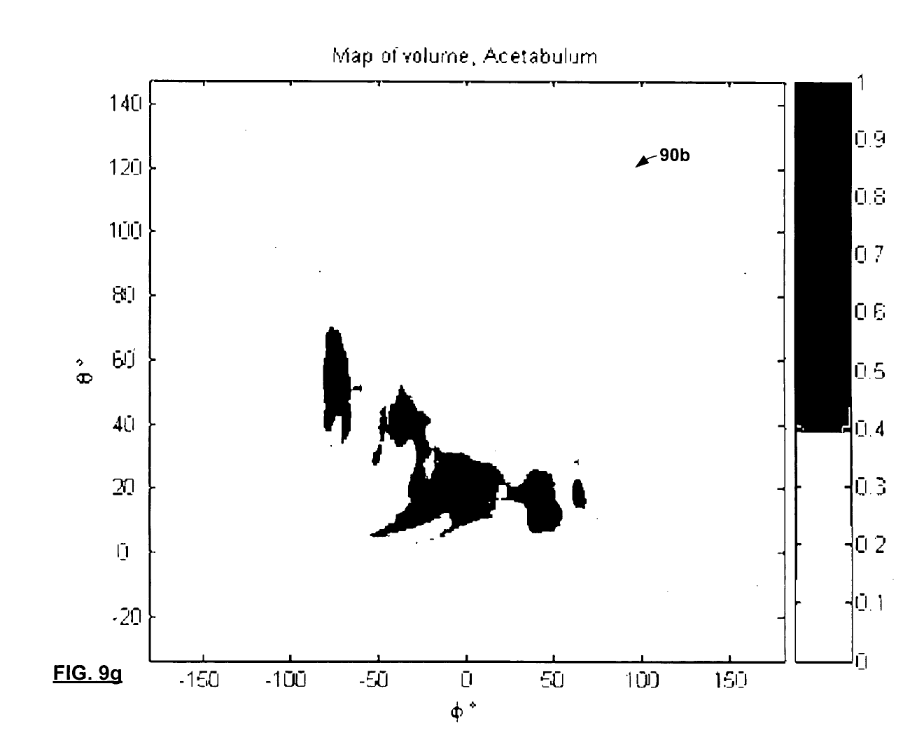

Referring to FIGS. 9a and 9b, the system can be used to make a variety of measurements on hip cartilage. After an individual acquisition, the system can provide its results in a number of different ways, such as in numeric form or as cartilage volume maps 90a, 90b. These types of results can allow a user to make qualitative and quantitative assessments about the condition of the hip cartilage. One suitable technique that is applicable to the hip is described in U.S. Pat. Application No. 20060002600, published on Jan. 5, 2006, and herein incorporated by reference.

The system can also be used to compare data from different acquisitions of the same patient over time. In this type of operation, the system preferably begins by registering the data sets relative to each other (step 124). To this end, the data sets preferably include part of the femur that lies below its neck. This additional bone image data helps to provide an accurate registration between the data sets, even though it is far from the region of interest in the images.

With the extra bone in the image, registration methods suitable for use with the knee can be used for the hip data. A variation field method can be used, for example, without initialization. Bone contour points are converted into splines, and the spline surfaces are registered using their initial position. Once the data sets are registered, the system can readily compare the state of the joints for the two acquisitions, such as by presenting the data sets as a difference map (step 126).

Comparing changes of cartilage over time can allow the progression of disease to be tracked. It can also be used to evaluate whether a therapy, such as the use of a pharmaceutical agent, is effective at slowing the progression of a disease such as osteoarthritis. And because the progression of osteoarthritis in the hip can be quite rapid, using the hip as a locus for evaluating pharmaceutical agents can provide results relatively quickly.

The present invention has now been described in connection with a number of specific embodiments thereof. However, numerous modifications which are contemplated as falling within the scope of the present invention should now be apparent to those skilled in the art. Therefore, it is intended that the scope of the present invention be limited only by the scope of the claims appended hereto. In addition, the order of presentation of the claims should not be construed to limit the scope of any particular term in the claims.

What is claimed is:

1. An MRI image processing method, comprising:
accessing a first MRI data set including a first plurality of two-dimensional planar MRI images of a first hip of a first patient,
wherein each of the images is positioned with respect to a virtual axis at a different angle so as to distribute the images axially around the virtual axis,
wherein the virtual axis is defined as an axis that runs from the fovea through the femoral neck of the femur of the first patient, and
segmenting bone and cartilage surfaces in the first MRI data set accessed in the step of accessing.

2. The method of claim 1 further including the step of measuring a cartilage characteristic based on results of the step of segmenting.

3. The method of claim 1 further including a step of accessing a second MRI data set acquired after the first data set and including another plurality of two-dimensional planar MRI images of the first hip of the first patient,
wherein each of the images passes through the virtual axis at a different angle so as to distribute the images axially around the virtual axis, and
segmenting bone and cartilage surfaces in the second MRI data set accessed in the step of accessing.

4. The method of claim 3 further including the step of spatially registering results of the steps of segmenting for the first and second MRI data sets.

5. The method of claim 4 further including the step of comparing results of the steps of segmenting to evaluate cartilage changes.

6. The method of claim 4 wherein the step of spatially registering takes place for at least part of the femur that is below the neck of the femur.

7. The method of claim 3 further including the step of comparing results of the steps of segmenting to evaluate cartilage changes.

8. The method of claim 7 further including the step of evaluating disease progression based on the step of comparing.

9. The method of claim 7 further including the step of evaluating a pharmaceutical effect on disease progression based on the step of comparing.

10. The method of claim 1 wherein the step of accessing accesses a plurality of images acquired with a water excitation sequence.

11. The method of claim 10 wherein the step of accessing accesses a plurality of MEDIC images.

12. The method of claim 1 wherein the step of accessing accesses a plurality of images acquired with a semi-flexible antenna.

13. The method of claim 1 wherein the step of accessing accesses plurality of images in which there is a forced internal rotation of the femoral neck.

14. The method of claim 1 wherein the virtual axis bisects the MRI images.

15. The method of claim 1 further including the steps of fitting a sphere to the femoral head of the first hip of the first patient and transforming data derived from the MRI data set into a polar coordinate system that is based on the sphere fitted in the step of fitting.

16. The method of claim 15 further including the step of expressing positions as a distance to the center of the sphere.

17. The method of claim 1 further including the steps of selectively reorienting the images in the first plurality of two-dimensional planar MRI images and assembling them into a three-dimensional image data set.

18. An MRI image processing apparatus, comprising:
an MRI data access module operative to access a first MRI data set including a first plurality of two-dimensional planar MRI images of a first hip of a first patient,
wherein each of the images is positioned with respect to a virtual axis at a different angle so as to distribute the images axially around the virtual axis,
wherein the virtual axis is defined as an axis that runs from the fovea through the femoral neck of the femur of the first patient, and
a segmentation module operative to segment bone and cartilage surfaces in the first MRI data set accessed by the MRI data access module.

19. The apparatus of claim 18 further including a registration module responsive to the segmentation module.

20. The apparatus of claim 18 further including an image comparison module responsive to at least the segmentation module.

21. The apparatus of claim 18 wherein the MRI data access module is directly responsive to an MRI acquisition system that employs a MEDIC sequence.

22. The apparatus of claim 18 wherein interconnections between elements include intermittent connections.

23. An MRI image processing apparatus, comprising:
means for accessing a first MRI data set including a first plurality of two-dimensional planar MRI images of a first hip of a first patient,
wherein each of the images is positioned with respect to a virtual axis at a different angle so as to distribute the images axially around the virtual axis,
wherein the virtual axis is defined as an axis that runs from the fovea through the femoral neck of the femur of the first patient, and
means for segmenting bone and cartilage surfaces in the first MRI data set accessed by the means for accessing.
